(19) United States
(12) Patent Application Publication
(10) Pub. No.: US 2012/0095098 A1
(43) Pub. Date: Apr. 19, 2012

(54) Bioavailability Enhancement Delivery System

(76) Inventor: Bhiku Patel, Chandler, AZ (US)

(21) Appl. No.: 12/906,419

(22) Filed: Oct. 18, 2010

(51) Int. Cl. A61K 31/202

(52) U.S. Cl. 514/560

(57) ABSTRACT

A composition for increasing the bioavailability of drugs in humans and animals comprising a microemulsion further comprising a first emulsifier, a second emulsifier, and an oil wherein the emulsifiers have individual HLB values of between about 10 and about 30, are in a ratio ranging from about 1:1 to about 4:1; and, the first and second emulsifier combination and oil are in a ratio of about 99:1 to about 9:1. A method for increasing the bioavailability of such a composition is also provided.

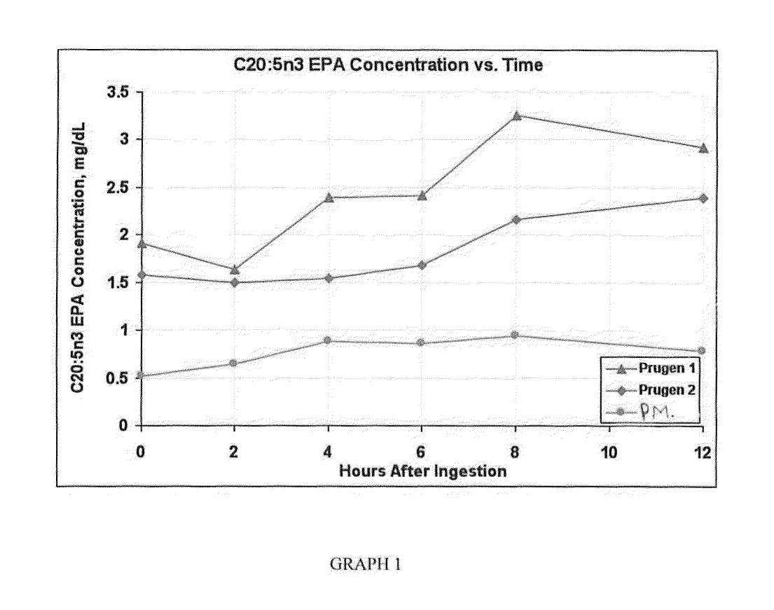

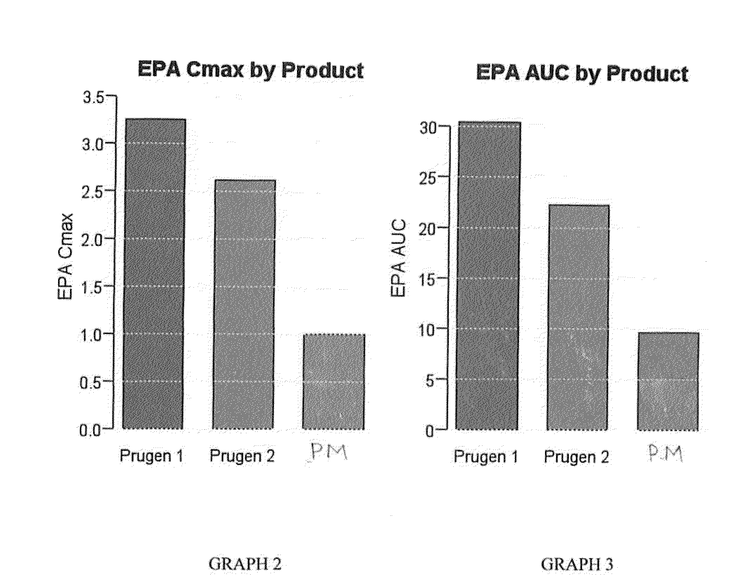

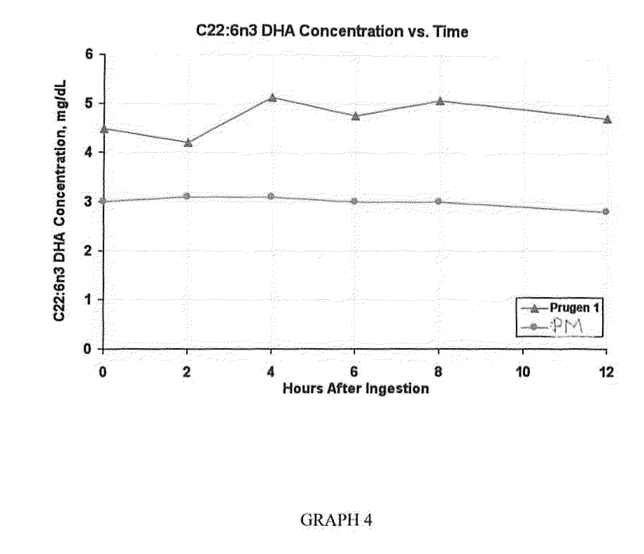

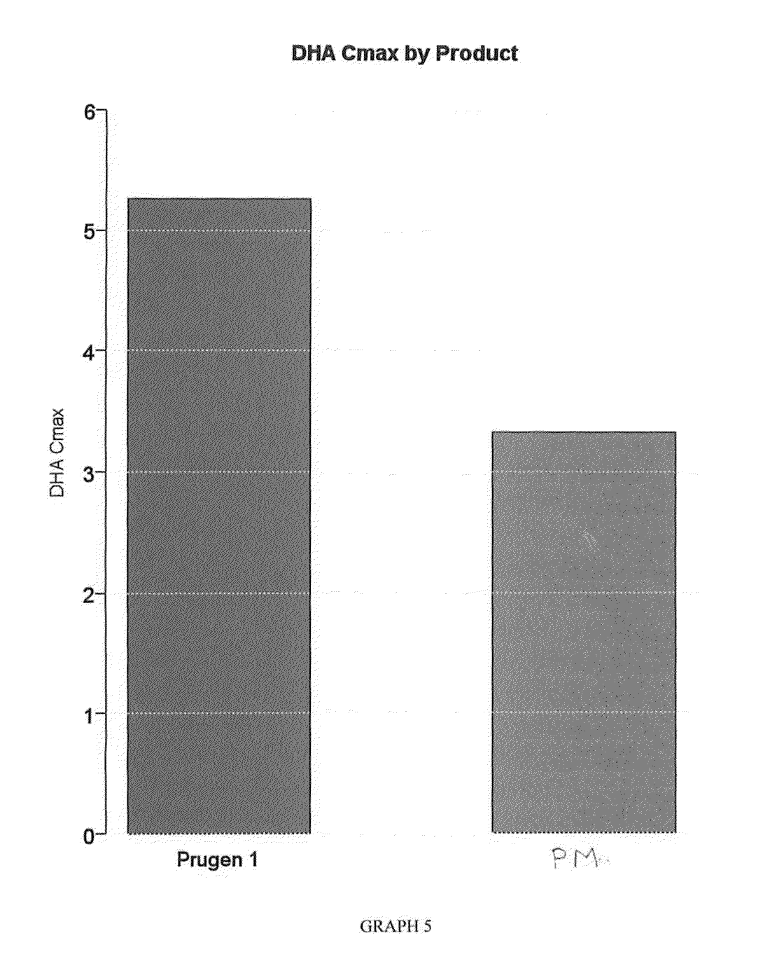

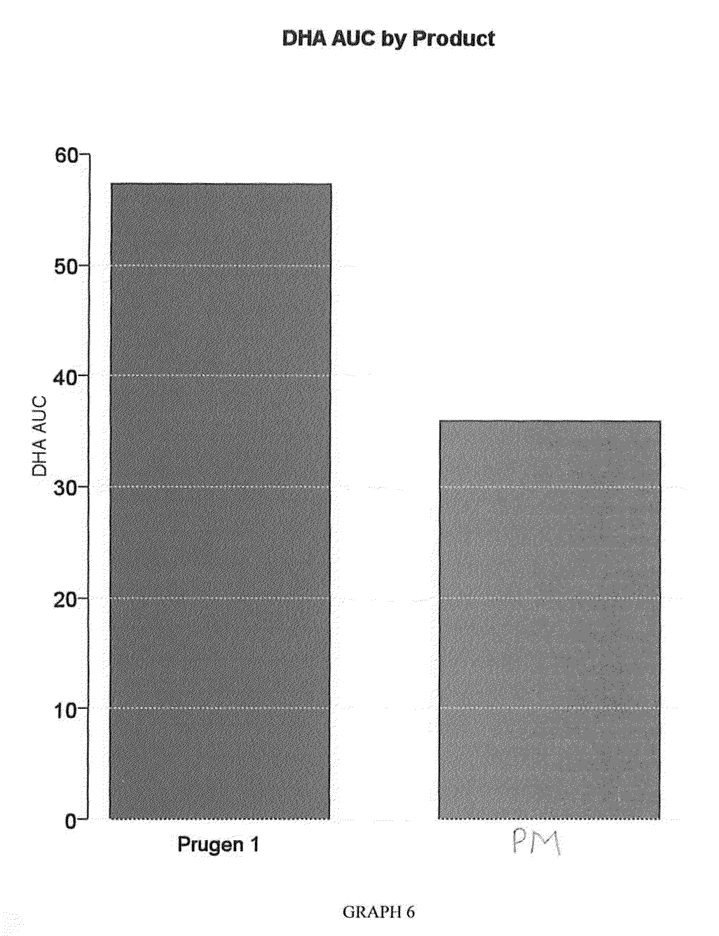

### I. TECHNICAL FIELD

The present invention relates to the delivery of oral pharmaceuticals (drugs), nutraceuticals, vitamins, minerals, and other health therapeutics consumed by humans or animals and, more particularly, to a microemulsion composition that enhances the bioavailability of such agents through increased dispersion and absorption.

### II. BACKGROUND OF THE INVENTION

Drug delivery is the method or process of administering a pharmaceutical compound to achieve a therapeutic effect in humans or animals. (Throughout, reference to drug also includes nutraceutical, vitamin, mineral, and other health related therapeutics.) Drug delivery systems can work by modifying drug release profile, absorption, distribution and elimination for the benefit of improving product efficacy and safety, as well as patient convenience and compliance. The most common routes of administration include the peroral (through the mouth), topical, transmucosal (nasal, buccal/sublingual, vaginal, ocular and rectal), intra-muscular injection, arterio/venous injection, and inhalation routes.

Historically, pharmaceuticals have primarily consisted of simple, fast-acting chemical compounds. More recently, however, formulations that control the rate and period of drug delivery have become increasingly common and complex.

Current methods of drug delivery exhibit specific problems that scientists are attempting to address. For example, many drugs' potencies and therapeutic effects are limited or otherwise reduced because of the partial degradation that occurs before they reach a desired target. This can lead to a need for increased dosages in an attempt to reach a therapeutically acceptable level of available drug. This, in turn, can lead to an increase in side effects and adverse events. To address these issues, researchers have recently focused efforts in the areas of micro- and nano-technology. While these areas may hold some promise, they tend to affect the chemical structure of the target drug in some manner, thereby creating yet another hurdle in delivering drugs.

Other prior art attempts to improve drug delivery that do not require alteration of the drug have included emulsions. Broadly speaking, an emulsion is a mixture of two or more immiscible (unblendable) liquids. Emulsions are lyophobic and generally unstable. Prior art drug delivery emulsion attempts have focused on macroemulsions which produce large, white droplets that tend to separate. They have also required heat and the input of considerable amounts of mechanical energy, which may be supplied by colloid mills, homogenizers, and ultrasonic generators. This raises the cost of preparation in a potentially less than desirable product.

Accordingly, there has been a long felt un-meet need to create an economical, stable emulsion based drug delivery system that overcomes the need for heat and mechanical energy in its preparation, while at the same increases the bioavailability of drugs without altering the drug itself, and while obtaining the goal of delivering the drug at the lowest therapeutically acceptable level. The present invention addresses this need through a microemulsion drug delivery system that distinguishes itself from the prior art by increasing dispersion and absorption of drugs, thereby increasing drug bioavailability at reduced dosage amounts while lowering the potential for systemic drug toxicity, all without altering the drug itself. The present invention further addresses the long felt un-met need of creating a microemulsion that does not depend on heat or mechanical energy in its preparation.

### III. OBJECTS AND ADVANTAGES OF THE PRESENT INVENTION

It is an object of the present invention to provide a microemulsion composition that enhances the bioavailability of a drug.

It is further an object of the present invention to provide a microemulsion composition that shortens the time of absorption of a drug.

It is further an object of the present invention to provide a finely dispersed drug.

It is further an object of the present invention to enable the reduction in the amount of a drug to be taken by a patient.

It is further an object of the present invention to provide an enhanced drug bioavailability delivery system that does not alter the target drug.

It is further an object of the present invention to produce a microemulsion based drug delivery system that does not require heat or mechanical energy in its preparation.

It is further an object of the present invention to provide a stable microemulsion drug delivery system.

It is further an object of the present invention to provide a more economical microemulsion based drug delivery system than has been seen in the prior art.

The advantages offered by the present invention include but are not limited to maximizing the amount of bioavailable drug while, at the same time, potentially lowering the required drug dosage without altering the target drug, all in a stable format.

The above objects and advantages are provided in the present invention—a stable microemulsion that does not require heat or mechanical energy in its preparation.

### IV. SUMMARY OF THE INVENTION

The present invention comprises a composition for increasing the bioavailability of a drug in humans and animals comprising a microemulsion produced by adding a first emulsifier and a second emulsifier in a ratio ranging from about 1:1 to about 4:1, preferably, the first emulsifier and the second emulsifier are mixed in a ratio of about 2:1, and an oil, with the final mixture of the first emulsifier and second emulsifier combination and the oil being in a ratio ranging from about 99:1 to about 9:1. Preferably, the ratio of the emulsifier combination and the oil is about 12.333:1. Preferably, the first emulsifier is polyoxyethylene sorbitan monooleate and the second emulsifier is tocopheryl polyethylene glycol succinate. The oil can be of any type of consumable oil, with currently known sources being animal, vegetable, marine-based, and algae oils. The oils can be used either singly or in combination with one another.

To determine the suitable range of surface area activity for the microemulsion, hydrophilic lipophilic balance (HLB) values are used. HLB values for emulsifiers range from about 2 to about 40. The Inventor discovered that emulsifiers in the range of about 10 to about 30 are suitable for use with the invention, with a range of about 12 to about 18 providing the best results.

The Inventor has also discovered that certain oils, due to their chemical structure, will not satisfactorily disperse if admixed in a one-step process. For these oils, a two-step process is required wherein the oil is added to one of the emulsifiers and then the other emulsifier is added to the first emulsifier-oil combination. Whether the oil is added to the first emulsifier or the second emulsifier depends on the oil's affinity for a particular emulsifier. In this process, the HLB ranges and combination ratio ranges of the final composition do not change.

To the mixture is added a target drug in a therapeutically acceptable amount that, by adjusting ratios within the HLB range, will solubilize within the mixture. The drug can be of any type. It was further discovered that, due to the chemical structure of certain drugs, an additional solubilizing agent may be required. Once the solubilized drug touches water, for instance, in the gastro-intestinal tract of a patient, it disperses in a fine microemulsion. The dispersed drug is then absorbed quickly due, in part, to the increased surface area created by the microemulsion and, in part, by the second emulsifier.

Using the inventive composition results in increased levels of drug blood stream concentrations. As measured by EPA levels, increases of about 250% over that of purified fish oil and about 400% over that of a prescription fish oil based medication, or about 0.253 mg/dl/hr versus 0.10 mg/dl/hr of purified fish oil and 0.06 mg/dl/hr for the prescription fish oil based medication based upon a gram intake have been demonstrated.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### V. BRIEF DESCRIPTION OF THE DRAWINGS

Graph Number 1 depicts EPA blood level Concentration vs Time (Hrs) for the inventive composition compared with purified fish oil alone and with a prescription fish oil based medication.

Graph Number 2 depicts the maximum EPA blood level concentration for the inventive composition compared with purified fish oil alone and with a prescription fish oil based medication.

Graph Number 3 depicts the maximum EPA AUC for the inventive composition compared with purified fish oil alone and with a prescription fish oil based medication

Graph Number 4 depicts DHA blood level Concentration vs Time (hrs) for the inventive composition compared with prescription fish oil based medication.

Graph Number 5 depicts the maximum DHA blood level concentration for the inventive composition compared with prescription fish oil based medication.

Graph Number 6 depicts the maximum DHA AUC for the inventive composition compared with a prescription fish oil based medication.

### VI. DETAILED DESCRIPTION OF THE PREFERRED EMBODIMENT

Before explaining the preferred embodiment of the present invention in detail, it is to be understood that the present invention is not limited in its application to the details of formulations and arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for the various agents and drug examples, they are approximate ranges and are not to be limiting except where noted otherwise.

The present invention addresses the problem of drug bioavailability through the use of emulsions. (As noted above, throughout, reference to drug also includes nutraceutical, vitamin, mineral, and other health related therapeutics.) More specifically, the present invention provides for the creation of a microemulsion to which drugs of any type can be solubilized and then, as described herein, dispersed and absorbed at rates not seen in the prior art. The advantages of microemulsions multi-fold. They include, without limitation, thermodynamic stability and, if the emulsifiers have been selected properly, will occur almost spontaneously without external mechanical energy and heat. Microemulsion droplets have a mean diameter range of approximately 6 to 100 nm and a narrow droplet size distribution. By contrast non-micro emulsions have broad droplet size distributions and are generally large and opaque.

To overcome prior art limitations in absorption and bioavailability of drugs, the Inventor has discovered that creating a finely dispersed microemulsion in turn creates an increase in drug surface area per volume, thereby enabling greater gastrointestinal uptake of the drug. Through the use of emulsifiers that do not create a permanent binding to the drug, the bioavailability of the drug is increased without altering the drug itself. Importantly, the Inventor has discovered that such a microemulsion can be made without the prior art limitations of heat and mechanical energy, thereby creating a stable microemulsion that can be produced at lower cost due to the energy savings.

To create a microemulsion adequate to provide the necessary results, the Inventor discovered that two emulsifiers within a specific HLB range added to a carrier oil are required. A first emulsifier should be a nonionic surfactant. There is a wide range of such emulsifiers from which to choose. Key to the selection is that the first emulsifier be suitable for human or animal ingestion (throughout, the terms consumption and ingestion are used interchangeable and mean to take orally.) The Inventor has discovered that the preferred first emulsifier is polyoxyethylene sorbitan monooleate.

A second emulsifier is also required. The Inventor has discovered that employing esterfied Vitamin E works well. While esterfied Vitamin E in its various forms may be employed in the invention, the Inventor has discovered that the preferred form is tocopheryl polyethylene glycol succinate: it is not only a solubilizer but an absorption enhancer.

Both of the preferred emulsifiers are well known in the industry. Polyoxyethylene sorbitan monooleate has been used for many years in the food and pharmaceutical industries. It is most commonly sold under the trade name polysorbate 80 and is widely available. It is approved by the U.S. Food and Drug Administration as an inactive ingredient and is well tolerated in oral compositions.

Tocopheryl polyethylene glycol succinate is sold by several companies but was first developed by the Eastman Company and sold under the trademark ‘Vitamin E TPGS NF.’ It was developed as a water soluble emulsifier to aid in the absorption of lipid-based drugs, such as cyclosporin. Since its invention, it has been used in many products.

However, until the present invention the combination use of polyoxyethylene sorbitan monooleate and tocopheryl polyethylene glycol succinate plus the addition of an oil to enhance the bioavailability of drugs has not been taught. In fact, such use could be seen as taught away from as conventional wisdom would hold that the use of an oil with these emulsifiers, particularly tocopheryl polyethylene glycol succinate, would saturate them, rendering them useless.

Individually, each of these emulsifiers will cause at least some dispersion but it is the heretofore unknown mixture of two emulsifiers and oil that creates the microemulsion necessary to increase the bioavailability of the drug. Using the inventive composition, a fine microemulsion is created that enables almost complete drug dispersion in water, a result unseen with either of the two emulsifiers separately or known in the prior art. The result is gained by the emulsion properties of the tocopheryl polyethylene glycol succinate to create a fine dispersion and the polyoxyethylene sorbitan monooleate to reduce further the interfacial tension that then enables an oil-in-water microemulsion of a level not heretofore seen in the art. The oil is important in that it creates an environment wherein the two emulsifiers can combine without interference and enable solubilization of the target drug.

When considering the selection of emulsifiers to use in combination to create a suitable microemulsion, the Inventor discovered that surface activity of the emulsifiers on the oil was an important element. Increasing the surface area of the oil enabled increased bioavailability but only to a point. Once surface area exceeds a certain value, no additional benefit is gained in absorption and, therefore, bioavailability. In fact, bioavailability can be decreased.

To determine the suitable range of surface area activity, the Inventor employed hydrophilic lipophilic balance (HLB) values. HLB is a widely accepted method for providing a measure of the surface activity of organic molecules. HLB values for emulsifiers range from about 2 to about 40. The Inventor discovered that emulsifiers in the range of about 10 to about 30 are suitable for use with the invention, with a range of about 12 to about 18 providing the best results.

In mixing of the two emulsifiers with oil, the ratios of the two emulsifiers and oil in creating an end product with maximum effectiveness at a minimum use of emulsifiers have been considered. The Inventor has discovered that an emulsifier ratio in the range of polyoxyethylene sorbitan monooleate:tocopheryl polyethylene glycol succinate (“emulsifier combination”) of about 1:1 to about 4:1 is useful. The preferred ratio is 2:1.

Considering the incentive composition from an HLB and ratio standpoint, the Inventor discovered the following: polyoxyethylene sorbitan monooleate has an HLB value of about 15 and tocopheryl polyethylene glycol succinate has an HLB value about 13. Thus, the average HLB value for the combination of the two emulsifiers is about 14 when they are found in a 1:1 polyethylene glycol succinate: tocopheryl polyethylene glycol succinate ratio and about 14.6 when they are in a 4:1 polyethylene glycol succinate: tocopheryl polyethylene glycol succinate ratio. Accordingly, the preferred HLB range is from about 14 to about 14.6 in the suitable ratio range of about 1:1 to about 4:1.

To the emulsifer combination is added a carrier oil. The oil can be of any type, with currently known sources being animal, vegetable, marine-based, and algae. The only requirement of the oil is that it is consumable.

In mixing the three components together, the emulsifier combination, which can be done as a separate step and added to the oil or individually added to the oil, the Inventor has discovered that the final oil:emulsifier combination should be about 99:1 to about 9:1. Levels above 99:1 do not allow adequate dispersion to take place and levels below 9:1 cause gelling of the oil:emulsifier combination. The preferred ratio is about 12.333:1.

The Inventor has also discovered that certain oils, due to their chemical structure, will not disperse in an acceptable manner if added in a one-step process. For these oils, a two-step process is required. For instance, the inventor discovered that due to its unique properties, oregano essential oil cannot be added directly into the base mixture a first emulsifier and a second emulsifier combination—it will not disperse—even by adjusting the ratios of the various components. The reason for this is that low molecular weight compounds, terpenes such as oregano essential oil, and their derivatives require a terpenoid type molecule first for dispersion (such as a flaxseed oil/second emulsifier solution). Otherwise, if put into solution simultaneously with the first emulsifier and the second emulsifier, before it is saturated or solubilized, it will bind to the second emulsifier and not disperse.

To the mixture of carrier oil and emulsifier is added a drug in a therapeutically acceptable amount. The drug can be of any type. It is solubilized in the inventive composition by adjusting the ratio of emulsifiers to drug within the acceptable HLB range, thereby creating a solubilized drug product that will disperse immediately upon impact with water, such as when in the gastro-intestinal tract of a patient and, in turn, absorbs quickly due to the fine microemulsion environment and second emulsifier. This, as demonstrated below, increases the bioavailability of the drug. The Inventor also discovered that certain drugs, due to their chemical structure, will not disperse fully without the aid of an additional solubilizer. The additional solubilizer can be of any type but the Inventor has discovered that polyethoxylated castor oil, polyethylene glycols, propylene glycol, fatty acids and esters, ethoxylated fatty acids and esters, alcohols, and their derivatives, either singly or in combination, will fully disperse most known drugs.

To demonstrate the effectiveness of the combination of these two emulsifiers and drugs, the Inventor developed an experiment to illustrate the increased bioavailability of a drug, using EPA and DHA as markers. The clinical protocol was developed by an FDA registered Clinical Organization and approved by an Internal Review Board (IRB). In the experiment, subjects were cleared of blood stream detectable levels of EPA and DHA. The Human subjects were then randomly given either the Inventive composition, a purified fish oil, or a fish oil-based prescription medication under physician supervision. All three products contained the same levels of DHA and EPA. In addition, the size, weight, and shape of all three products were the same. Blood level readings for both EPA and DHA were then taken at 2, 4, 6, 8, 10, and 12 hours.

Turning to Graph Number 1, it can be seen that EPA levels for the inventive composition were significantly above those for both the purified fish oil and the prescription medication at every reading. The following table 1 illustrates the rate of blood level concentration (mg/dl/hr) for each of the three test materials:

TABLE 1

| Inventive Composition | Purified Fish oil | Prescription Medication |
|---|---|---|
| 0.253 | 0.10 | 0.06 |

Turning to Graph Number 2, it can be seen that the maximum blood concentration (Cₘₐₓ) for the Inventive Composition EPA is also greater than that of the purified fish oil and the prescription medication. The following table 2 shows the Cₘₐₓ (mg/dl) for each of the three test materials:

TABLE 2

| Inventive Composition | Purified Fish oil | Prescription Medication |
|---|---|---|
| 3.27 | 2.62 | 1.00 |

Graph Number 3 illustrates the Area Under Curve (AUC) for the three test materials and, again demonstrates the superiority of the Inventive Composition. Table 3 shows the average AUC₀₋₁₂ (mg/dl/hr) for the three test materials:

TABLE 3

| Inventive Composition | Purified Fish oil | Prescription Medication |
|---|---|---|
| 30.40 | 22.30 | 9.70 |

DHA readings were then taken to further demonstrate the Inventive Composition effectiveness. Turning to Graph 4, it can be seen that DHA levels for the inventive composition were significantly above those of the prescription medication at every reading.

Turning to Graph Number 5, it can be seen that the maximum blood concentration (Cₘₐₓ) for the Inventive Composition DHA is also greater than that of the prescription medication. The following table 4 shows the Cₘₐₓ (mg/dl) for the two test materials:

TABLE 4

| Inventive Composition | Prescription Medication |
|---|---|
| 5.26 | 3.34 |

Graph Number 6 illustrates the Area Under Curve (AUC) for the two test materials and, again demonstrates the superiority of the Inventive Composition. The AUC₀₋₁₂ for the Inventive Composition is 57.3 mg/dl/hr. and for the prescription medication is 33.0 mg/dl/hr.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

I claim:

1. A composition for increasing the bioavailability of drugs in humans and animals comprising a microemulsion further comprising a first emulsifier, a second emulsifier, and an oil.

2. The composition of claim 1 wherein a drug is then solubilized in the first emulsifier, second emulsifier, and oil.

3. The drug of claim 2 wherein drug includes the group comprising pharmaceuticals, drugs, nutraceuticals, vitamins, and minerals.

4. The composition of claim 1 wherein the first emulsifier and the second emulsifier are in a ratio ranging from about 1:1 to about 4:1.

5. The composition of claim 1 wherein the first emulsifier and the second emulsifier are in a ratio of about 2:1.

6. The composition of claim 1 wherein the first emulsifier and the second emulsifier, in combination, and oil are in a ratio ranging from about 99:1 to about 9:1.

7. The composition of claim 1 wherein the first emulsifier and the second emulsifier, in combination, and oil are in a ratio ranging of about 12.333:1.

8. The composition of claim 1 wherein the first emulsifier is polyoxyethylene sorbitan monooleate.

9. The composition of claim 1 wherein the second emulsifier is tocopheryl polyethylene glycol succinate.

10. The composition of claim 1 wherein the oil is selected, either singly or in combination from the group comprising animal, vegetable, marine-based, and algae oils.

11. The composition of claim 1 wherein the first emulsifier and the second emulsifier have individual HLB values in the range of about 10 to about 30.

12. The composition of claim 1 wherein the first emulsifier and the second emulsifier have individual HLB values in the range of about 12 to about 18.

13. The composition of claim 1 wherein the first emulsifier and second emulsifier, when in combination, have an HLB value in the range of about 14 to about 14.6.

14. The composition of claim 1 further including a solubilizing agent.

15. The solubilizing agent of claim 14 selected from the group, either singly or in combination, comprising polyethoxylated castor oil, polyethylene glycols, propylene glycol, fatty acids and esters, ethoxylated fatty acids and esters, alcohols, and their derivatives.

16. A microemulsion composition for increasing the bioavailability of drugs in humans and animals comprising polyoxyethylene sorbitan monooleate and a second emulsifier in a ratio ranging from about 1:1 to about 4:1 and an oil, wherein the polyoxyethylene sorbitan monooleate and the second emulsifier, in combination, is in a ratio to the oil ranging from about 99:1 to about 9:1.

17. The composition of claim 16 wherein the polyoxyethylene sorbitan monooleate and the second emulsifier are in a ratio of about 2:1.

18. The composition of claim 16 wherein the polyoxyethylene sorbitan monooleate and the second emulsifier are in a ratio of about 12.333:1.

19. The composition of claim 16 wherein the oil is selected, either singly or in combination from the group comprising animal, vegetable, marine-based, and algae oils.

20. The composition of claim 16 wherein the polyoxyethylene sorbitan monooleate and the second emulsifier have individual HLB values in the range of about 10 to about 30.

21. The composition of claim 16 wherein the polyoxyethylene sorbitan monooleate and the second emulsifier have individual HLB values in the range of about 12 to about 18.

22. The composition of claim 16 wherein the polyoxyethylene sorbitan monooleate and second emulsifier, when in combination, have an HLB value in the range of about 14 to about 14.6.

23. The composition of claim 16 further including a drug solubilized in the polyoxyethylene sorbitan monooleate, second emulsifier, and oil.

24. The drug of claim 23 wherein drug is selected from the group comprising pharmaceuticals, drugs, nutraceuticals, vitamins, and minerals.

25. The composition of claim 16 further including a solubilizing agent.

26. The solubilizing agent of claim 25 selected from the group, either singly or in combination, comprising polyethoxylated castor oil, polyethylene glycols, propylene glycol, fatty acids and esters, ethoxylated fatty acids and esters, alcohols, and their derivatives.

27. A microemulsion composition for increasing the bioavailability of drugs in humans and animals comprising a first emulsifier and tocopheryl polyethylene glycol succinate in a ratio ranging from about 1:1 to about 4:1 and an oil, wherein the first emulsifier and the tocopheryl polyethylene glycol succinate, in combination, is in a ratio to the oil ranging from about 99:1 to about 9:1.

28. The composition of claim 27 wherein the first emulsifier and the tocopheryl polyethylene glycol succinate are in a ratio of about 2:1.

29. The composition of claim 27 wherein the first emulsifier and the tocopheryl polyethylene glycol succinate are in a ratio of about 12.333:1.

30. The composition of claim 27 wherein the oil is selected, either singly or in combination from the group comprising animal, vegetable, marine-based, and algae oils.

31. The composition of claim 27 wherein the first emulsifier and the tocopheryl polyethylene glycol succinate have individual HLB values in the range of about 10 to about 30.

32. The composition of claim 27 wherein the first emulsifier and the tocopheryl polyethylene glycol succinate have individual HLB values in the range of about 12 to about 18.

33. The composition of claim 27 wherein the first emulsifier and tocopheryl polyethylene glycol succinate, when in combination, have an HLB value in the range of about 14 to about 14.6.

34. The composition of claim 27 further including a drug solubilized in the mixture of the first emulsifier, tocopheryl polyethylene glycol succinate, and oil.

35. The composition of claim 35 wherein the drug is selected from the group comprising pharmaceuticals, drugs, nutraceuticals, vitamins, and minerals.

36. The composition of claim 27 further including a solubilizing agent.

37. The solubilizing agent of claim 36 selected from the group, either singly or in combination, comprising polyethoxylated castor oil, polyethylene glycols, propylene glycol, fatty acids and esters, ethoxylated fatty acids and esters, alcohols, and derivatives.

38. A composition for increasing the bioavailability of drugs in humans and animals comprising a microemulsion further comprising a first emulsifier, a second emulsifier, an oil and a drug.

39. The composition of claim 38 wherein the first emulsifier and the second emulsifier are in a ratio ranging from about 1:1 to about 4:1.

40. The composition of claim 38 wherein the first emulsifier and the second emulsifier are in a ratio of about 2:1.

41. The composition of claim 38 wherein the first emulsifier and the second emulsifier, in combination, and oil are in a ratio ranging from about 99:1 to about 9:1.

42. The composition of claim 38 wherein the first emulsifier and the second emulsifier, in combination, and oil are in a ratio ranging of about 12.333:1.

43. The composition of claim 38 wherein the first emulsifier is polyoxyethylene sorbitan monooleate.

44. The composition of claim 38 wherein the second emulsifier is tocopheryl polyethylene glycol succinate.

45. The composition of claim 38 wherein the oil is selected, either singly or in combination from the group comprising animal, vegetable, marine-based, and algae oils.

46. The composition of claim 38 wherein the drug includes the group comprising pharmaceuticals, drugs, nutraceuticals, vitamins, and minerals.

47. The composition of claim 38 wherein the first emulsifier and the second emulsifier have individual HLB values in the range of about 10 to about 30.

48. The composition of claim 38 wherein the first emulsifier and the second emulsifier have individual HLB values in the range of about 12 to about 18.

49. The composition of claim 38 wherein the first emulsifier and second emulsifier, when in combination, have an HLB value in the range of about 14 to about 14.6.

50. The composition of claim 38 further including a solubilizing agent.

51. The solubilizing agent of claim 50 selected from the group, either singly or in combination, comprising polyethoxylated castor oil, polyethylene glycols, propylene glycol, fatty acids and esters, ethoxylated fatty acids and esters, alcohols, and their derivatives.

52. A method for increasing the bioavailability of a drug comprising the steps of:
combining a first emulsifier, a second emulsifier and an oil, the first emulsifier and second emulsifier, in combination, being in a ratio ranging from about 1:1 to about 4:1 with a resulting HLB range of about 10 to about 30, and the ratio of the first emulsifier and second emulsifier, in combination, and oil being from about 99:1 to about 9:1;
solubilizing a drug in the combination of first emulsifier, a second emulsifier and oil, thereby creating a microemulsion;
providing the microemulsion to a patient;
having the microemulsion come into contact with water;
dispersing the drug; and,
having the drug absorbed.

53. The method of claim 52 including the step of selecting the first emulsifier as polyoxyethylene sorbitan monooleate.

54. The method of claim 52 including the step of selecting the second emulsifier as tocopheryl polyethylene glycol succinate.

55. The method of claim 52 including the step of selecting the oil, either singly or in combination, from the group comprising animal, vegetable, marine-based, and algae oils.

56. The further step of claim 52 further including the step of selecting the drug, either singly or in combination, from the group comprising pharmaceuticals, drugs, nutraceuticals, vitamins, and minerals.

57. The method of claim 52 further including the further step of adding a solubilizing agent to further enhance the solubilization of a drug.

58. The further step of claim 57 including the further step of selecting the solubilizing agent from the group, either singly or in combination, comprising polyethoxylated castor oil, polyethylene glycols, propylene glycol, fatty acids and esters, ethoxylated fatty acids and esters, alcohols, and derivatives.
